(19) United States
(12) Patent Application Publication
(10) Pub. No.: US 2025/0120575 A1
(43) Pub. Date: Apr. 17, 2025

(54) TUBE MANAGEMENT DEVICES AND RELATED METHODS OF REMOVABLY COUPLING MEDICAL TUBES

(71) Applicant: Boston Scientific Scimed, Inc., Maple Grove, MN (US)

(72) Inventors: Austin Grant JOHNSON, Worcester, MA (US); Colby HARRIS, Norfolk, MA (US); Jennifer LEWITZKY, Lexington, MA (US)

(73) Assignee: Boston Scientific Scimed, Inc., Maple Grove, MN (US)

(21) Appl. No.: 18/904,536

(22) Filed: Oct. 2, 2024

Related U.S. Application Data
(60) Provisional application No. 63/589,369, filed on Oct. 11, 2023.

(51) Int. Cl. A61B 1/00

(52) U.S. Cl. CPC A61B 1/0014

(57) ABSTRACT

A device for tube management includes a base. The base includes a structure configured to transition the device between an extended configuration and a coiled configuration upon receiving an application of a force to the device. The device also includes a coating applied to the base. In the extended configuration, the device includes a channel configured to receive a tube. In the coiled configuration, the device is configured to circumferentially surround a medical device shaft to removably couple the tube to the medical device shaft.

### CROSS-REFERENCE TO RELATED APPLICATIONS

This application claims the benefit of priority of U.S. Provisional Patent Application No. 63/589,369, filed on Oct. 11, 2023, the entirety of which is incorporated herein by reference.

### TECHNICAL FIELD

The disclosure relates generally to tube management devices. More specifically, aspects of the disclosure pertain to tube management devices and related methods of use for removably coupling medical tubes.

### BACKGROUND

Accessory devices used in conjunction with a medical device, such as an endoscope, to perform a medical procedure may often include one or more tubes or wires inserted into a body lumen of a patient via a working channel of the endoscope that extends interiorly through the endoscope shaft. However, in some examples, the accessory device may additionally or alternatively include one or more tubes or wires configured to extend along an exterior of the endoscope shaft. For example, an exterior tube may serve as an additional working channel through which instruments, irrigation, and/or suction may be delivered or objects may be removed from the body lumen.

Conventionally, tape or a similar adhesive mechanism has been used to couple the tubes or wires to the exterior of the endoscope shaft. For example, tape may be applied around an exterior tube and the shaft at one or more positions along a length of the shaft. The coupling may prevent the exterior tube from free floating when the exterior tube is inserted along with the shaft into the body lumen to help mitigate any view obstruction and/or tissue trauma during a medical procedure.

### SUMMARY

A device for tube management may include a base. The base may include a structure configured to transition the device between an extended configuration and a coiled configuration upon receiving an application of a force to the device. The device may also comprise a coating applied to the base. In the extended configuration, the device may include a channel configured to receive a tube. In the coiled configuration, the device may be configured to circumferentially surround a medical device shaft to removably couple the tube to the medical device shaft.

In any of the exemplary devices disclosed herein, the extended configuration may be a substantially linear configuration, and the coiled configuration may be a substantially curved configuration.

In some aspects, the channel may be a recess in an interior surface of the device. In the coiled configuration, the device may be configured to circumferentially surround the medical device shaft and the tube received within the channel.

In other aspects, the channel may protrude from an exterior surface of the device. The channel may be a partially opened channel. The partially opened channel may include an opening configured to receive the tube that is formed from a pair of arms extending outwardly and perpendicularly from the exterior surface of the device. The channel may be an enclosed channel. The enclosed channel may include a tubular member extending outwardly and substantially perpendicularly from the exterior surface of the device, and the tubular member may include a tubular member lumen configured to receive the tube. The channel may be configured to receive the tube, while the device is in the extended configuration and in the coiled configuration

In further aspects, the coating may include a first coating and a second coating different from the first coating. The first coating may be applied to one or more surfaces of the base configured to be exposed to a body lumen when the medical device shaft to which the tube is removably coupled via the device is partially inserted into the body lumen. The second coating may be applied to one or more surfaces of the base configured to contact at least one of the medical device shaft or the tube.

In additional aspects, when the device is in the coiled configuration, the structure of the base may be configured to transition the device from the coiled configuration back to the extended configuration to enable repositioning of the device relative to at least one of the medical device shaft or the tube. The base may include metal, and the coating may include rubber, plastic, or a combination of rubber and plastic.

A medical system may include a first medical tube, a second medical tube separate from the first medical tube, and a tube management device configured to removably couple the first medical tube to the second medical tube. The tube management device may include a base. The base may include a structure configured to transition the tube management device between an extended configuration and a coiled configuration. The base may also include a coating applied to the base. In the coiled configuration, the tube management device may be configured to circumferentially surround the first medical tube and the second medical tube to removably couple the first medical tube to the second medical tube.

Any of the exemplary medical systems disclosed herein may include any of the following features. The tube management device may further include a channel configured to receive one of the first medical tube or the second medical tube. The channel may be a recess in an interior surface of the tube management device. When the tube management device is in the coiled configuration, the structure of the base may be configured to transition the tube management device from the coiled configuration back to the extended configuration to enable repositioning of the tube management device relative to at least one of the first medical tube or the second medical tube.

A method for removably coupling medical tubes may include positioning a device relative to a first medical tube and a second medical tube separate from the first medical tube, where the device may be in an extended configuration. The method may also include applying a first force to the device to transition the device from the extended configuration to a coiled configuration, such that the device circumferentially surrounds at least one of the first medical tube or the second medical tube to removably couple the first medical tube to the second medical tube.

In any of the exemplary methods disclosed herein, the positioning of the device may include: receiving one of the first medical tube or the second medical tube within a channel of the device to couple the one of the first medical tube or the second medical tube to the device; and positioning the device coupled to the one of the first medical tube or the second medical tube relative to the other of the one of the first medical tube or the second medical tube.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIGS. 1A-1H depict various views of an exemplary medical system and components thereof, including a first tube management device.

FIGS. 2A-2D depict various views of a second tube management device.

FIGS. 3A-3D depict various views of a third tube management device.

FIGS. 4A-4D depict various views of a fourth tube management device.

FIG. 5 depicts an example method for removably coupling medical tubes.

### DETAILED DESCRIPTION

As briefly mentioned above, an accessory device used in conjunction with a medical device, such as an endoscope, for performing a medical procedure may include one or more tubes or wires configured to extend along an exterior of the endoscope shaft, including a distal portion of the shaft that is inserted into a body lumen of the patient. For example, an exterior tube may serve as an additional working channel through which instruments, irrigation, and/or suction may be delivered or objects may be removed from the body lumen. In other examples, over the scope devices may include shafts, tubes, or similar structures that may extend along an exterior of the endoscope shaft.

Conventionally, tape or a similar adhesive mechanism may be used to couple the tubes or wires to the exterior of the endoscope shaft. For example, tape may be applied around an exterior tube and the endoscope shaft at one or more positions along a length of the shaft. The coupling may prevent the exterior tube from free floating when the exterior tube is inserted along with the endoscope shaft into the body lumen to help mitigate any view obstruction and/or tissue trauma during a medical procedure.

However, the tape application process is inefficient. Adhesives, like tape, are by nature sticky. As a result, as an operator is removing the tape from packaging and manipulating the tape for application, the tape may erroneously stick to unintended objects (e.g., other devices, materials, packaging, etc.) or to the medical device and/or the accessory device at an unintended position. Thus, the application process may be time consuming due to additional handling precautions taken by the operator to prevent such erroneous actions. Once the tape is applied to couple the tube of the accessory device to the endoscope shaft at a first position, the tape may be difficult to remove for repositioning to a second position, if needed, and/or may not be reusable for application at the second position (e.g., the adhesive qualities of the tape may be significantly reduced due to the removal). Additionally, such erroneous application and/or repositioning of the tape may potentially cause damage to the medical device (e.g., endoscope), accessory device, or other unintended objects that the tape comes into contact with during the application or repositioning process. Further, the idea of inserting tape into the body may generally be off-putting to patients.

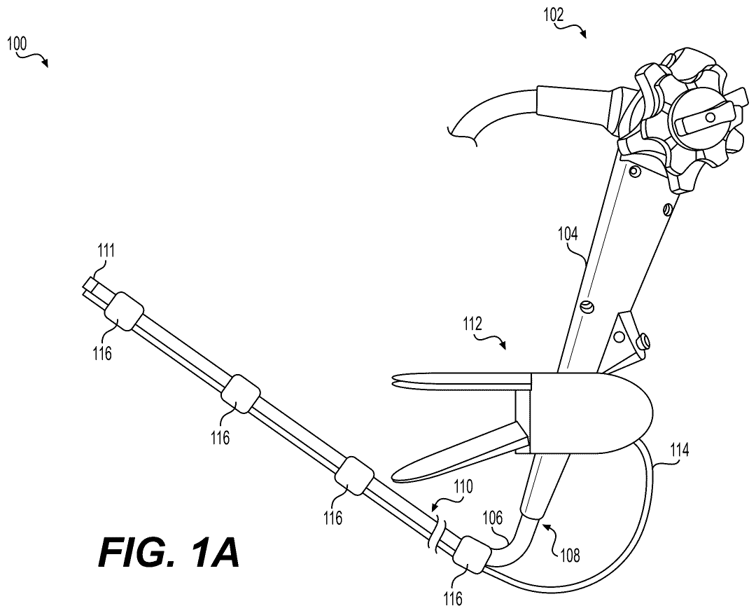

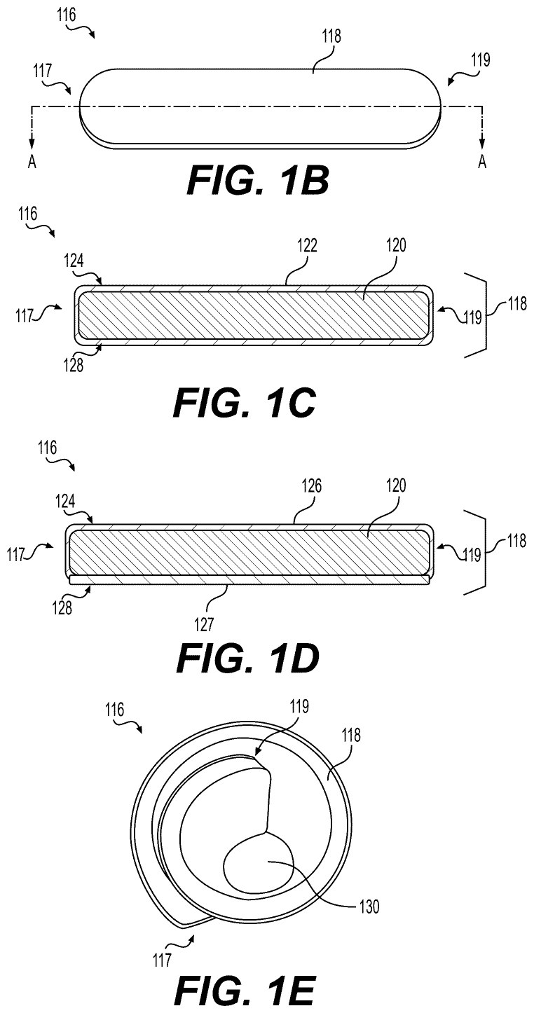

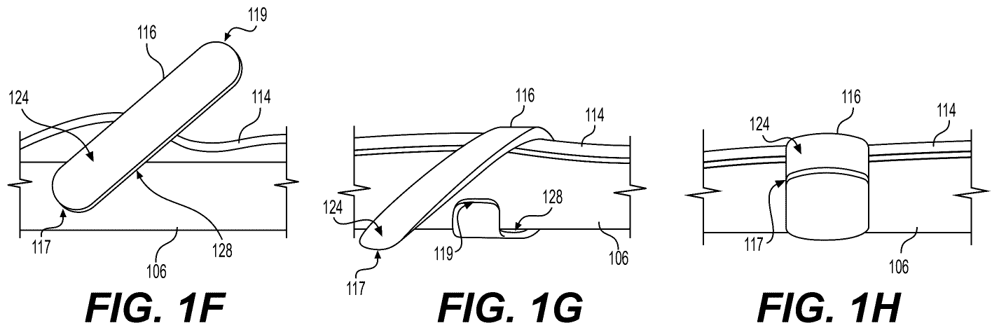

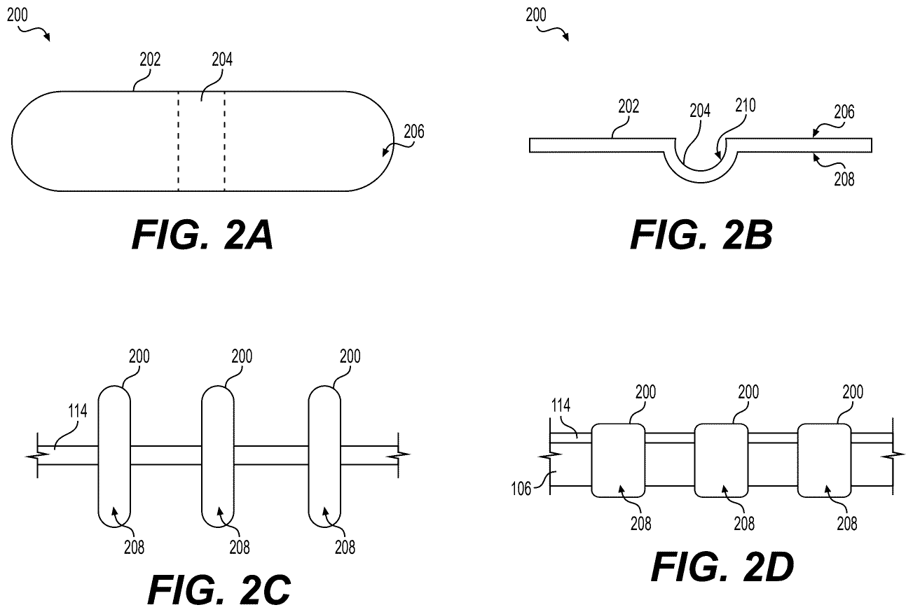

Therefore, aspects of this disclosure are directed to tube management devices and related methods of use for removably coupling two or more separate medical tubes to one another, such as an exterior tube of an accessory device to a shaft of a medical device. An exemplary tube management device may include a multi-layer body comprising a base and a coating. The tube management device may include an extended configuration and a coiled configuration, where the base may include a structure configured to transition the tube management device between the extended configuration and the coiled configuration upon an application of force (e.g., applied by an operator) to the tube management device. For example, the tube management device, in the extended configuration, may be positioned relative to the medical device shaft and tube, and an operator may apply a first force to the tube management device to cause the structure to transition the tube management device to the coiled configuration such that the tube management device circumferentially surrounds at least the medical device shaft. Based on a configuration of the tube management device, the tube management device may circumferentially surround both the medical device shaft and the tube when in the coiled configuration, such as the configuration of the tube management device shown in FIGS. 1A-1H and FIGS. 2A-2D.

If the tube management device needs to be repositioned along the shaft, the structure of the base enables the tube management device to quickly and easily transition back from the coiled configuration to the extended configuration for repositioning. The structure can facilitate repeated transitions of the tube management device back and forth between the extended configuration and the coiled configuration without affecting or degrading the coupling functionality of the tube management device.

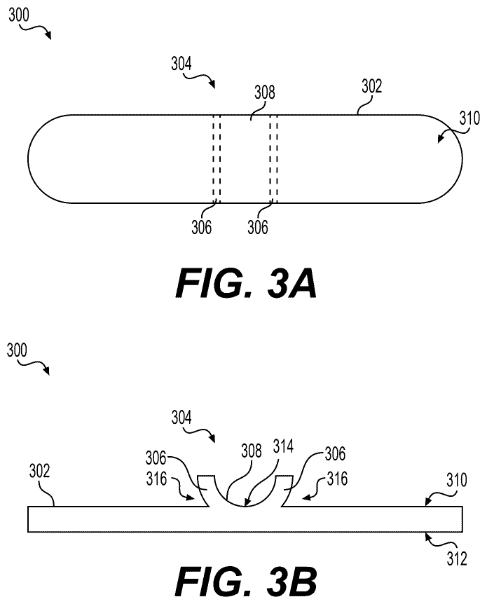

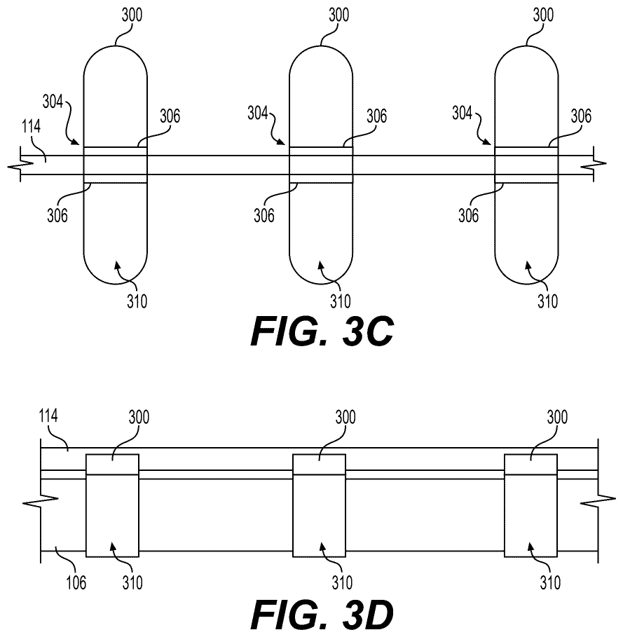

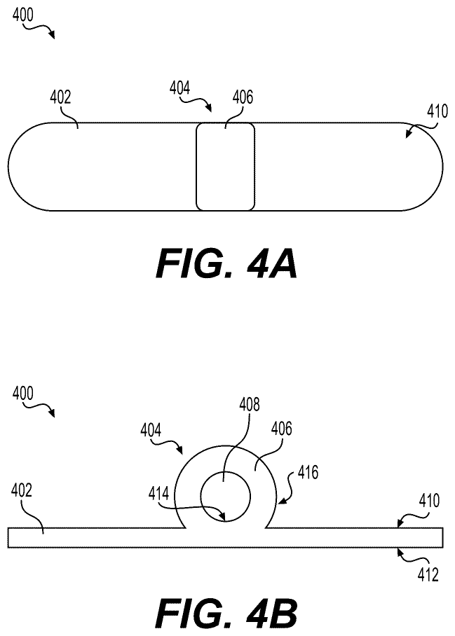

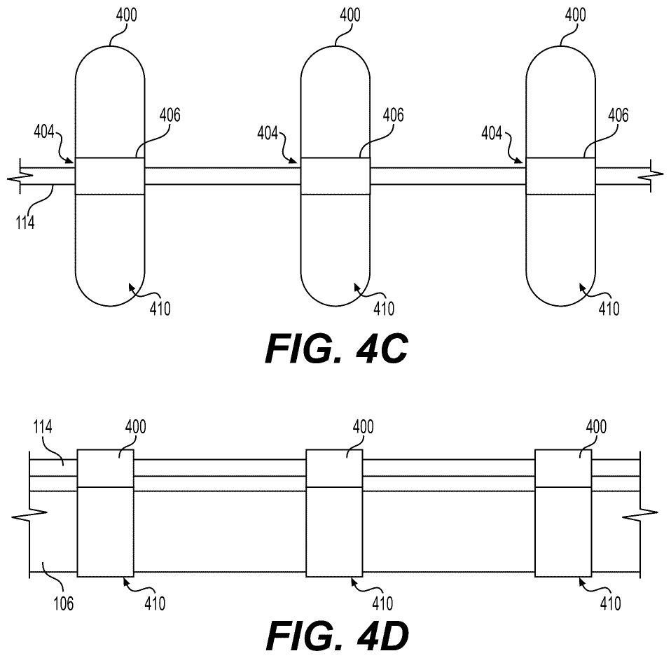

Some configurations of the tube management device may further include a channel that is configured to receive the tube. In one example configuration and as described with reference to FIGS. 2A-D below, the channel may be a recess in an interior surface of a body of the tube management device. In another example configuration, the channel protrudes from an exterior surface of the body of the tube management device. The protruding channel may be a partially opened channel as described with reference to FIGS. 3A-D below or an enclosed channel as described with reference to FIGS. 4A-D below. The channel of the tube management device may help reduce or eliminate compression of the tube such that a full inner diameter of the tubing may be utilized during the medical procedure.

The coating of the tube management device may be applied to surfaces of the base and/or the channel. In some examples, a combination of coatings may be applied. For example, a first coating may be applied to a first set of surfaces of the base and/or the channel that will be exposed to the body lumen when the shaft, to which the tube is removably coupled via the tube management device, is partially inserted into the body lumen. This first coating may be comprised of a hydrophobic and/or lubricious material to facilitate the insertion and to help reduce tissue trauma. A second coating may be applied to a second set of surfaces of the base and/or the channel that contact the shaft when the base circumferentially surrounds the shaft in the coiled configuration or contact the tube when received by the channel. The second coating may be different from the first coating. For example, the second coating may be comprised of a high friction material configured to help increase a friction, adherence, or grip of the base to the shaft and/or the tube.

FIG. 1A depicts an exemplary medical system 100. Medical system 100 may include a first medical device 102, a second medical device 112, and one or more first tube management devices 116 configured to removably couple components of first medical device 102 and second medical device 112 to one another. While four first tube management devices 116 are illustrated in medical system 100 of FIG. 1A, more or fewer first tube management devices 116 may be utilized. A number of first tube management devices 116 utilized may be based on a size or length of first medical device 102, a type of medical procedure being performed (e.g., a function to be performed by second medical device 112), or the like.

First medical device 102 may be an endoscope or other type of scope, such as a bronchoscope, ureteroscope, duodenoscope, gastroscope, endoscopic ultrasonography (“EUS”) scope, colonoscope, laparoscope, arthroscope, cystoscope, aspiration scope, sheath, catheter, or other medical tube. First medical device 102, as shown in FIG. 1A, may include a handle 104 and a shaft 106. A proximal portion 108 of shaft 106 may be attached to and extend distally from handle 104, and at least a distal portion 110 of shaft 106 may be inserted into a body lumen of a patient during a medical procedure. In some examples, a size (e.g., at least an outer diameter) of shaft 106 may be constant along a length of shaft 106. In other examples, the size of shaft 106 may vary along a length of shaft 106.

Distal tip 111 at a distalmost end of distal portion 110 may include distal openings of one or more working channels and/or an imaging system, including one or more illumination devices and/or one or more visualization devices, of first medical device 102. The working channels may extend along an interior of shaft 106 and may be configured to supply fluid, apply suction, and/or deliver devices, tubes, and/or wires to the treatment site via the distal openings. Illumination devices (e.g., one or more LEDs, optical fibers, and/or other illuminators) of the imaging system may be configured to illuminate areas of the patient's base during the procedure to, e.g., facilitate imaging. Visualization devices (e.g., one or more cameras, one or more image sensors, endoscopic viewing elements, optical assemblies including one or more image sensors and one or more lenses, etc.) of the imaging system may be configured to capture images during the medical procedure.

Second medical device 112 may be a device separate from first medical device 102 that is used together with first medical device 102 to perform the medical procedure. For example, second medical device 112 may be an accessory device, such as an endoscopic suturing device. In some examples, and as shown in FIG. 1A, second medical device 112 may be fixed or otherwise coupled to first medical device 102 (e.g., via handle 104) to facilitate operation of both first medical device 102 and second medical device 112 by an operator.

Second medical device 112 may include a tube 114 or other shaft that extends distally from second medical device 112. In other examples, second medical device 112 may be comprised solely of tube 114 (e.g., second medical device 112 may be a medical tube). A portion of tube 114 may be configured to extend along an exterior of shaft 106, including along at least distal portion 110 of shaft 106 that is inserted into the body lumen to perform the procedure. In some examples, and as shown in FIG. 1A, tube 114 may also at least partially extend along proximal portion 108 of shaft 106. Tube 114 may provide an exterior working channel in addition to the one or more working channels extending within the interior of shaft 106. For example, instruments, devices, irrigation, suction or the like may be delivered to the body lumen via tube 114 and/or objects may be removed from the body lumen via tube 114. In other examples, second medical device 112 may be an over-the-scope device (e.g., a suturing device, clip device, banding device, etc.), which may include a shaft extending along an exterior of shaft 106 (e.g., for carrying control members, such as wires). Although the term “tube” 114 is used herein, it will be appreciated that the disclosed devices may instead include other types of shafts, cables, or wires. In some examples, a size (e.g., at least an outer diameter) of tube 114 may be constant along a length of tube 114. In other examples, the size of tube 114 may vary along a length of tube 114.

Each first tube management device 116, when in a coiled configuration as shown in FIG. 1A, may removably couple tube 114 to shaft 106 at a position along shaft 106 to facilitate insertion into and prevent tube 114 from free-floating within the body lumen. First tube management device 116 may include two configurations: an extended configuration and the coiled configuration. FIG. 1B illustrates a top perspective view of first tube management device 116 in the extended configuration. FIGS. 1C and 1D illustrate cross-sectional views of first tube management device 116 along line A-A shown in FIG. 1B, where FIG. 1C depicts a first multi-layer configuration of first tube management device 116 and FIG. 1D depicts a second multi-layer configuration of first tube management device 116. FIG. 1E illustrates first tube management device 116 in the coiled configuration.

As shown in FIG. 1B, in the extended configuration, a body 118 of first tube management device 116 may be substantially straight or linear from a first end 117 to second end 119 along a longitudinal axis of body 118. As shown in FIG. 1E, in the coiled configuration, body 118 may be substantially curved such that a channel 130 is formed for enclosing one or more objects, such as shaft 106 and tube 114. In some examples, and as shown in FIG. 1E, a portion of body 118 may overlap with itself in the coiled configuration. For example, a first portion of body 118 including first end 117 may extend over (e.g., wrap or curl around) a second portion of body 118 including second end 119 to form channel 130.

Referring to FIG. 1B and FIG. 1E concurrently, body 118 may be sized to accommodate shaft 106 and tube 114. For example, a length of body 118 from first end 117 to second end 119 may be of a length such that, in the coiled configuration shown in FIG. 1E, an inner diameter of channel 130 formed to enclose shaft 106 and tube 114 is greater than an outer diameter of shaft 106 and an outer diameter of tube 114 combined. When more than one first tube management device 116 is utilized, as shown in FIG. 1A, each of first tube management devices 116 may be the same. In other examples, when an outer diameter of tube 114 and/or shaft 106 varies along a length of tube 114 and/or shaft 106 respectively, a length of body 118 of one or more first tube management devices 116 may be different from one another to accommodate the varied outer diameter of tube 114 and/or shaft 106, respectively. Although body 118 of first tube management device 116 is illustrated as being generally rectangular in shape with rounded ends, this disclosure is not so limited. In other examples, body 118 may be circular, ovular, elliptical, or square, among other shapes.

Referring to FIG. 1C, body 118 of first tube management device 116 may be comprised of a plurality of layers (e.g., may be a multi-layer body). The layers may include a base 120 and a coating 122. Base 120 may be an inner or interior layer comprised of metal, such as stainless steel, or plastic. Base 120 may include a structure configured to transition first tube management device 116 between the extended configuration shown in FIG. 1B and the coiled configuration shown in FIG. 1E. For example, similar to a slap bracelet, the structure may be a bistable structure having two stable equilibrium states, where the structure may rest in either of the two states. The two states may be mechanical shapes, and particularly an extended state or a coiled state. Accordingly, when the structure of base 120 transitions between the extended state and the coiled state, first tube management device 116 may correspondingly transition between the extended configuration and the coiled configuration.

In some examples, when the structure is in the extended state, body 118 may be bent in a first direction about a central longitudinal axis of body 118, such that sides of body 118 extend away from the central longitudinal axis of body 118 in a first direction. For example, an exterior surface 124 of body 118 may have a concave shape and an interior surface 128 of body 118 may have a convex shape in a cross-section that is perpendicular to the central longitudinal axis of body 118. When the structure is in the coiled state, body 118 may have a different shape. For example, body 118 may be flat or may be bent in a second direction (opposite the first direction) about the central longitudinal axis of body 118, such that sides of body 118 extend away from the central longitudinal axis of body 118 in a second direction. In the coiled state, exterior surface 124 may have a flat or convex shape in a cross-section that is perpendicular to the central longitudinal axis of body 118. In the coiled state, interior surface 128 may have a flat or concave shape in a cross-section that is perpendicular to the central longitudinal axis of body 118.

The structure of base 120 may transition between the extended state and the coiled state in response to a force applied to first tube management device 116 by an operator. One example force may include contacting a portion of first tube management device 116, including one of first end 117 or second end 119, against shaft 106 and/or tube 114 with sufficient force (e.g., by slapping against). Another example force may include applying a bending action to first end 117 and/or second end 119 of the first tube management device 116 (e.g., by pushing first end 117 and/or second end 119 inward toward center of body 118) to bend interiorly.

Continuing to refer to FIG. 1C, coating 122 may be applied to surfaces of base 120 such that coating 122 surrounds base 120 and forms an outer layer of body 118. Coating 122 may include silicone, rubber, or plastic. In some examples, and particularly when base 120 is comprised of metal, base 120 may include sharp edges. Coating 122 may cover or otherwise provide protection to the body lumen from the sharp edges to help reduce trauma to the body lumen. Additionally, coating 122 may help to increase a friction, adherence, or grip of body 118 to shaft 106 and tube 114 when body 118 circumferentially surrounds shaft 106 and tube 114 in the coiled configuration. Such increased friction, adherence, or grip may help prevent first tube management device 116 from sliding or otherwise falling off.

Exterior surface 124 of body 118 may include surface portions of body 118 that may be exposed to the body lumen when shaft 106, to which tube 114 is removably coupled via first tube management device 116, is partially inserted into the body lumen. In some examples, exterior surface 124 may include both a surface along a length of body 118 and surfaces along a height of body 118 at each of first end 117 and second end 119, as at least one of first end 117 or second end 119 may be exposed to the body lumen when in the coiled configuration, as described in detail below. Interior surface 128 of body 118 may include portions of body 118 that may contact shaft 106 when first tube management device 116 circumferentially surrounds at least shaft 106 in the coiled configuration. In some examples, and as shown in FIG. 1C, a same coating 122 may be applied to both exterior surface 124 and interior surface 128 of body 118.

In other examples, and as shown in FIG. 1D, a first coating 126 may be applied to exterior surface 124 and a second coating 127 different from first coating 126 may be applied to interior surface 128. First coating 126 may be comprised of a hydrophobic and/or lubricious material to facilitate the insertion of shaft 106 with tube 114 removably coupled thereto by first tube management device 116, and to help reduce tissue trauma. Second coating 127 may be comprised of a high friction material to increase a friction, adherence, or grip of body 118 to shaft 106 and tube 114 when body 118 circumferentially surrounds shaft 106 and tube 114 in the coiled configuration. Such increased friction, adherence, or grip may help prevent first tube management device 116 from sliding or otherwise falling off.

FIGS. 1F-1H depict an example method of use of first tube management device 116 to removably couple tube 114 to shaft 106. FIGS. 1F and 1G depict a front view of shaft 106 and tube 114 as first tube management device 116 is being applied. FIG. 1H depicts a rear view of shaft 106 and tube 114 (e.g., opposite of front view) after application of first tube management device 116. Referring back to FIG. 1F, tube 114 configured to extend along an exterior of shaft 106 may be free-floating prior to application of first tube management device 116. An operator may wish to couple tube 114 to shaft 106 at a position along shaft 106 using first tube management device 116. To do so, operator may position first tube management device 116, in the extended configuration, relative to a portion of tube 114 and shaft 106 at a desired position along shaft 106, as shown in FIG. 1F.

As a force is applied to first tube management device 116, the structure of base 120 transitions first tube management device 116 from the extended configuration to the coiled configuration, as shown in FIG. 1G. For example, interior surface 128 of a portion of body 118 including second end 119 initially contacts and begins to enclose (e.g., wrap or curl around) shaft 106 and tube 114 as body 118 substantially curves along the length from second end 119 to first end 117. An example force applied may include contacting, by an operator, second end 119 of first tube management device 116 against shaft 106 and/or tube 114 with sufficient force (e.g., by slapping against). Another example force applied may include applying, by an operator, a bending action to second end 119 (e.g., by pushing second end 119 inward toward a center of body 118) to bend interiorly. As shown in FIG. 1H, first tube management device 116 in the coiled configuration may entirely surround shaft 106 and tube 114 to removably couple tube 114 to shaft 106. Depending on a length of body 118 of first tube management device 116 relative to an outer diameter of shaft 106 and an outer diameter of tube 114, a portion of first tube management device 116 may overlap with itself. For example, and as shown in FIG. 1H, interior surface 128 of a portion of first tube management device 116 that includes first end 117 may overlap and contact an exterior surface 124 of another portion of first tube management device 116 that includes second end 119.

In some examples, the operator may have to reposition first tube management device 116 along shaft 106 (e.g., move from a first position to a second position). In such examples, the structure of base 120 in body 118 enables first tube management device 116 to quickly and easily transition from the coiled configuration shown in FIG. 1H back to the extended configuration shown in FIG. 1F for repositioning. For example, an operator may lift or pull first end 117 outward and away from shaft 106 to expose second end 119, and then may continue to lift or pull both first end 117 and second end 119 outward and away from shaft 106 with sufficient force (e.g., until a length of body 118 from first end 117 to second end 119 extends along a same plane). Such force may cause the structure of base 120 to transition first tube management device 116 back to the extended configuration. The structure can facilitate repeated transitions of first tube management device 116 back and forth between the extended configuration and the coiled configuration without affecting or degrading the coupling functionality of first tube management device 116.

While the specific application of first tube management device 116 described herein is associated with coupling a tube 114 of second medical device 112 to shaft 106 of first medical device 102 for insertion into a body lumen of a patient, first tube management device 116 is not limited to this application. For example, first tube management device 116 may be used for removably coupling any two medical tubes to one another.

FIGS. 2A-2D depict various views of a second tube management device 200. FIG. 2A depicts a top view of second tube management device 200 in an extended configuration. FIG. 2B depicts a side view of second tube management device 200 in the extended configuration. FIG. 2C depicts a top view of a plurality of second tube management devices 200 having received tube 114. FIG. 2D depicts a side view of second tube management devices 200 of FIG. 2C applied to shaft 106 in a coiled configuration to removably couple tube 114 to shaft 106.

Referring concurrently to FIGS. 2A and 2B, second tube management device 200 may include a body 202. Body 202 may be similar to body 118 of first tube management device 116. For example, an inner layer of body 202 may include base 120 having the structure configured to transition second tube management device 200 between the extended configuration and a coiled configuration. Additionally, an outer layer of body 202 may include coating 122 on both an interior surface 206 and an exterior surface 208 of body 202. Alternatively, the outer layer of body 202 may include first coating 126 on exterior surface 208 (including end surfaces) and second coating 127 on interior surface 206. However, different from body 118, a portion of body 202 may include a channel 204.

Channel 204 may be a recess in interior surface 206 of body 202. In some examples, channel 204 may be positioned mid-way along a length (e.g., at a center) of body 202. In other examples, channel 204 may be positioned off-center. Channel 204 may extend a width of body 202, approximately perpendicularly to a longitudinal axis of body 202. A surface 210 of channel 204 may include a same coating 122 or second coating 127 applied to interior surface 206 of body 202. Channel 204 may be maintained when second tube management device 200 is in both the extended configuration and the coiled configuration. In some examples, and as shown in FIG. 2B, a portion of exterior surface 208 of body 202 corresponding to a location of the recess may minimally protrude when in the extended configuration. In other examples, exterior surface 208 of body 202 may remain substantially linear when in the extended configuration.

Channel 204 may be sized to accommodate tube 114. For example, a size of channel 204 may be based on a size (e.g., an outer diameter) of tube 114. In some examples, channel 204 may be sized such that a portion of tube 114 placed or positioned within channel 204 is secured to prevent tube 114 from slipping or otherwise falling out of channel 204 (e.g., to secure tube 114 to second tube management device 200).

In the extended configuration, second tube management device 200 may be configured to receive tube 114 via channel 204 prior to coupling tube 114 to shaft 106. For example, an operator may place or position a portion of tube 114 within channel 204 of second tube management device 200, when second tube management device 200 is in the extended configuration, to couple tube 114 to second tube management device 200.

In some examples, and as shown in FIG. 2C, the operator may place or position a different portion of tube 114 within each channel 204 of a plurality of second tube management devices 200. In some examples, and as shown in FIGS. 2C and 2D, each of second tube management devices 200 may be the same. In other examples, when an outer diameter of tube 114 and/or shaft 106 varies along a length of tube 114 and/or shaft 106 respectively, a size of each channel 204 and/or length of body 202 of second tube management devices 200 may be different from one another to accommodate the varied outer diameter of tube 114 and/or shaft 106, respectively.

Second tube management devices 200, each in the extended configuration and having tube 114 coupled thereto, as shown in FIG. 2C, may then be positioned relative to shaft 106 at a desired position along shaft 106. As a force is applied to each of second tube management devices 200, the structure of base 120 in body 202 transitions each second tube management device 200 from the extended configuration to the coiled configuration, as shown in FIG. 2D. For example, interior surface 206 of body 202 contacts and encloses (e.g., wraps or curls around) shaft 106 as body 202 substantially curves along a length of body 202 in the coiled configuration. Shaft 106 and tube 114 may be enclosed in a channel formed by body 202, similar to channel 130 formed by body 118 in FIG. 1E. In some examples, the force may be applied individually and sequentially to each second tube management device 200. In other examples, the force may be applied simultaneously to two or more of second tube management devices 200.

In some examples, the operator may have to reposition one of second tube management devices 200 along shaft 106. In such examples, the structure of base 120 in body 202 enables second tube management device 200 to quickly and easily transition from the coiled configuration shown in FIG. 2D back to the extended configuration shown in FIG. 2C for repositioning. For example, an operator may lift or pull both ends of body 202 outward and away from shaft 106 with sufficient force (e.g., until a length of body 202 extends along a same plane). Such force may cause the structure of base 120 to transition second tube management device 200 back to the extended configuration. The structure can facilitate repeated transitions of second tube management device 200 back and forth between the extended configuration and the coiled configuration without affecting or degrading the coupling functionality of second tube management device 200.

Because tube 114 is received within channel 204 that is formed in interior surface 206 of body 202, in the coiled configuration shown in FIG. 2D, interior surface 206 of each second tube management device 200 may be configured to surround both shaft 106 and tube 114. Coating 122 or second coating 127 on interior surface 206 of body 202, including surface 210 of channel 204, may help to improve a friction, adherence, or grip of body 202 of second tube management device 200 to shaft 106 and tube 114. Exterior surface 208 of each second tube management device 200 may be exposed to a body lumen when the coupled shaft 106 and tube 114 are partially inserted into the body lumen during a medical procedure. Coating 122 or first coating 126 on exterior surface 208 may help to facilitate the insertion and to help reduce tissue trauma. Further, accommodation of tube 114 within channel 204 may help at least mitigate a compression of tube 114 when tube 114 is coupled to shaft 106 such that a full inner diameter of tube 114 may be utilized (e.g., as an additional working channel) during the medical procedure.

FIGS. 3A-3D depict various views of a third tube management device 300. FIG. 3A depicts a top view of third tube management device 300 in an extended configuration. FIG. 3B depicts a side view of third tube management device 300 in the extended configuration. FIG. 3C depicts a top view of a plurality of third tube management devices 300 having received tube 114. FIG. 3D depicts a side view of third tube management devices 300 of FIG. 3C applied to shaft 106 in a coiled configuration to removably couple tube 114 to shaft 106.

Referring concurrently to FIGS. 3A and 3B, third tube management device 300 may include a body 302. Body 302 may be similar to body 118 of first tube management device 116. For example, an inner layer of body 302 may include base 120 having the structure configured to transition third tube management device 300 between the extended configuration and a coiled configuration. Additionally, an outer layer of body 302 may include coating 122 on both an interior surface 312 and an exterior surface 310 of body 302. Alternatively, the outer layer of body 302 may include first coating 126 on exterior surface 310 (including end surfaces) and second coating 127 on interior surface 312. However, different from body 118, a portion of body 302 may include a channel 304.

Channel 304 may protrude outwardly from an exterior surface 310 of body 302. In some examples, channel 304 may be positioned mid-way along a length (e.g., at a center) of body 302. In other examples, channel 304 may be positioned off-center. Channel 304 may extend a width of body 302, approximately perpendicularly to a longitudinal axis. In some examples, body 302 and channel 304 may be formed or manufactured as a single component. In other examples, body 302 and channel 304 may be formed or manufactured as separate components, where channel 304 is affixed or otherwise coupled to body 302. Channel 304 may be maintained when third tube management device 300 is in both the extended configuration and the coiled configuration.

Channel 304 may be a partially opened channel. For example, channel 304 may include an opening 308 formed from a pair of arms 306 extending outwardly and perpendicularly from exterior surface 310. A surface 314 of channel 304 (e.g., interior surfaces of arms 306) may include a same coating 122 or second coating 127 applied to interior surface 312 of body 202. Exterior surfaces 316 of arms 306 may include a same coating 122 or first coating 126 applied to exterior surface 310 of body 302.

Channel 304 may be sized to accommodate tube 114. For example, a size of channel 304 may be based on a size (e.g., an outer diameter) of tube 114. In some examples, channel 304 may be sized such that a portion of tube 114 placed or positioned within channel 304 is secured to prevent tube 114 from slipping or otherwise falling out of channel 304 (e.g., to couple tube 114 to third tube management device 300). For example, arms 306 may be slightly flexible so as to bend to receive tube 114. Arms 306 may be sufficiently resilient that channel 304 holds tube 114 with an interference.

Third tube management device 300 may be configured to receive tube 114 via channel 304 in either the extended or coiled configuration. As one example, when in the extended configuration, third tube management device 300 may be configured to receive tube 114 via channel 304 prior to coupling tube 114 to shaft 106. For example, an operator may place or position a portion of tube 114 within channel 304 of third tube management device 300, when third tube management device 300 is in the extended configuration, to couple tube 114 to third tube management device 300.

In some examples, and as shown in FIG. 3C, the operator may place or position a different portion of tube 114 within each channel 304 of a plurality of third tube management devices 300. In other examples, when an outer diameter of tube 114 and/or shaft 106 varies along a length of tube 114 and/or shaft 106 respectively, a size of each channel 304 and/or length of body 302 of third tube management devices 300 may be different from one another to accommodate the varied outer diameter of tube 114 and/or shaft 106, respectively.

Third tube management devices 300, each in the extended configuration and having tube 114 coupled thereto as shown in FIG. 3C, may then be positioned relative to shaft 106 at a desired position along shaft 106. As a force is applied to each of third tube management devices 300, the structure of base 120 in body 302 transitions each third tube management device 300 from the extended configuration to the coiled configuration, as shown in FIG. 3D. For example, interior surface 312 of body 302 contacts and encloses (e.g., wraps or curls around) shaft 106 as body 302 substantially curves along a length of body 302 in the coiled configuration. Shaft 106 may be enclosed in a channel formed by body 302, similar to channel 130 formed by body 118 in FIG. 1E. In some examples, the force may be applied individually and sequentially to each third tube management device 300. In other examples, the force may be applied simultaneously to two or more of third tube management devices 300.

Alternatively, third tube management devices 300 may be configured to receive tube 114 via channel 304 when third tube management devices 300 are in the coiled configuration after being applied to shaft 106. For example, third tube management devices 300 may first be applied to shaft 106, and then third tube management devices, now in the coiled configuration, may receive tube 114 via channel 304 to removably couple tube 114 to shaft.

In some examples, the operator may have to reposition one of third tube management devices 300 along shaft 106. In such examples, the structure of base 120 in body 302 enables third tube management device 300 to quickly and easily transition from the coiled configuration shown in FIG. 3D back to the extended configuration shown in FIG. 3C for repositioning. For example, an operator may lift or pull both ends of body 302 outward and away from shaft 106 with sufficient force (e.g., until a length of body 302 extends along a same plane). Such force may cause the structure of base 120 to transition third tube management device 300 back to the extended configuration. The structure can facilitate repeated transitions of third tube management device 300 back and forth between the extended configuration and the coiled configuration without affecting or degrading the coupling functionality of third tube management device 300.

Coating 122 or second coating 127 on interior surface 312 of body 302 and surface 314 of channel 304 may help to improve a friction, adherence, or grip of body 202 of second tube management device 200 to shaft 106 and tube 114, respectively. Exterior surface 310 of each third tube management device 300 may be exposed to a body lumen when the coupled shaft 106 and tube 114 are partially inserted into the body lumen during a medical procedure. Coating 122 or first coating 126 on exterior surface 310 may help to facilitate the insertion and to help reduce tissue trauma. Further, accommodation of tube 114 within channel 304 may help mitigate or eliminate a compression of tube 114 when tube 114 is coupled to shaft 106 such that a full inner diameter of tube 114 may be utilized (e.g., as an additional working channel) during the medical procedure.

FIGS. 4A-4D depict various views of a fourth tube management device 400. FIG. 4A depicts a top view of fourth tube management device 400 in an extended configuration. FIG. 4B depicts a side view of fourth tube management device 400 in the extended configuration. FIG. 4C depicts a top view of a plurality of fourth tube management devices 400 having received tube 114. FIG. 4D depicts a side view of fourth tube management devices 400 of FIG. 4C applied to shaft 106 in a coiled configuration to removably couple tube 114 to shaft 106.

Referring concurrently to FIGS. 4A and 4B, fourth tube management device 400 may include a body 402. Body 402 may be similar to body 118 of first tube management device 116. For example, an inner layer of body 402 may include base 120 having the structure configured to transition fourth tube management device 400 between the extended configuration and a coiled configuration. Additionally, an outer layer of body 402 may include coating 122 on both an interior surface 412 and an exterior surface 410 of body 402. Alternatively, the outer layer of body 402 may include first coating 126 on exterior surface 410 (including end surfaces) and second coating 127 on interior surface 412. However, different from body 118, a portion of body 402 may include a channel 404.

Channel 404 may protrude outwardly from exterior surface 410 of body 402. In some examples, channel 404 may be positioned mid-way along a length (e.g., at a center) of body 402. In other examples, channel 404 may be positioned off-center.

Channel 404 may extend a width of body 402, approximately perpendicularly to a central longitudinal device of body 402. In some examples, body 402 and channel 404 may be formed or manufactured as a single component. In other examples, body 402 and channel 404 may be formed or manufactured as separate components, where channel 404 is affixed or otherwise coupled to body 402. Channel 404 may be maintained in both the extended configuration and the coiled configuration of fourth tube management device 400.

Channel 404 may be an enclosed channel. For example, channel 404 may include a tubular member 406 extending outwardly and substantially perpendicularly from exterior surface 410 of body 402. Tubular member 406 may include a tubular member lumen 408. A surface 414 of tubular member lumen 408 may include a same coating 122 or second coating 127 applied to interior surface 412 of body 202. An exterior surface 416 of tubular member 406 may include a same coating 122 or first coating 126 applied to exterior surface 410 of body 402.

Tubular member lumen 408 may be sized to accommodate tube 114. For example, a size of tubular member lumen 408 may be based on a size (e.g., an outer diameter) of tube 114. In some examples, tubular member lumen 408 may be sized such that tube 114 is able to be moved or fed through tubular member lumen 408 when force is applied to tube 114 (e.g., by an operator). Otherwise, when no force is applied, tube 114 may be closely fitted (e.g., interference fitted via friction) within tubular member lumen 408 to prevent tube 114 from unintentionally sliding or otherwise falling out of tubular member lumen 408 (e.g., to couple tube 114 to fourth tube management device 400).

Fourth tube management device 400 may be configured to receive tube 114 via tubular member lumen 408 of channel 404 in either the extended or coiled configuration. As one example, when in the extended configuration, fourth tube management device 400 may be configured to receive tube 114 prior to coupling tube 114 to shaft 106. For example, an operator may feed or slide a first end of tube 114 into and through tubular member lumen 408, when fourth tube management device 400 is in the extended configuration, to secure tube 114 to fourth tube management device 400.

In some examples, and as shown in FIG. 4C, the operator may sequentially feed or slide the first end of tube 114 into and through each tubular member lumen 408 of a plurality of fourth tube management devices 400. In some examples, and as shown in FIGS. 4C and 4D, each of fourth tube management devices 400 may be the same. In other examples, when an outer diameter of tube 114 and/or shaft 106 varies along a length of tube 114 and/or shaft 106 respectively, a size of each tubular member lumen 408 and/or length of body 202 of the plurality of fourth tube management devices 400 may be different from one another to accommodate the varied outer diameter of tube 114 and/or shaft 106, respectively.

Fourth tube management devices 400, each in the extended configuration and having tube 114 coupled thereto as shown in FIG. 4C, may then be positioned relative to shaft 106 at a desired position along shaft 106. As a force is applied to each of fourth tube management devices 400, the structure of base 120 in body 402 transitions each fourth tube management device 400 from the extended configuration to the coiled configuration, as shown in FIG. 4D. For example, interior surface 412 of body 402 contacts and encloses (e.g., wraps or curls around) shaft 106 as body 402 substantially curves along a length of body 402 in the coiled configuration. Shaft 106 may be enclosed in a channel formed by body 202, similar to channel 130 formed by body 118 in FIG. 1E. In some examples, the force may be applied individually and sequentially to each fourth tube management device 400. In other examples, the force may be applied simultaneously to two or more of fourth tube management devices 400.

Alternatively, fourth tube management devices 400 may be configured to receive tube 114 fed or slid into and through each tubular member lumen 408 when fourth tube management devices 400 are in the coiled configuration after being applied to shaft 106. For example, fourth tube management devices 400 may first be applied to shaft 106, and then tube 114 may be fed or slid into and through each tubular member lumen 408 of fourth tube management devices 400 by the operator to removably couple tube 114 to shaft 106.

In some examples, the operator may have to reposition one of fourth tube management devices 400 along shaft 106. In such examples, the structure of base 120 in body 402 enables fourth tube management device 400 to quickly and easily transition from the coiled configuration shown in FIG. 4D back to the extended configuration shown in FIG. 4C for repositioning. For example, an operator may lift or pull both ends of body 402 outward and away from shaft 106 with sufficient force (e.g., until a length of body 402 extends along a same plane). Such force may cause the structure of base 120 to transition fourth tube management device 400 back to the extended configuration. The structure can facilitate repeated transitions of fourth tube management device 400 back and forth between the extended configuration and the coiled configuration without affecting or degrading the coupling functionality of fourth tube management device 400.

Coating 122 or second coating 127 on interior surface 412 of body 402 and surface 414 of tubular member lumen 408 may help to improve a friction, adherence, or grip of body 402 of fourth tube management device 400 to shaft 106 and tube 114, respectively. Exterior surface 410 of body 402 and exterior surface 416 of tubular member 406 may be exposed to a body lumen when the coupled shaft 106 and tube 114 are partially inserted into the body lumen during a medical procedure. Coating 122 or first coating 126 on exterior surface 410 and exterior surface 416 may help to facilitate the insertion and to help reduce tissue trauma. Further, accommodation of tube 114 within tubular member lumen 408 may help mitigate or eliminate a compression of tube 114 when tube 114 is coupled to shaft 106 such that a full inner diameter of tube 114 may be utilized (e.g., as an additional working channel) during the medical procedure.

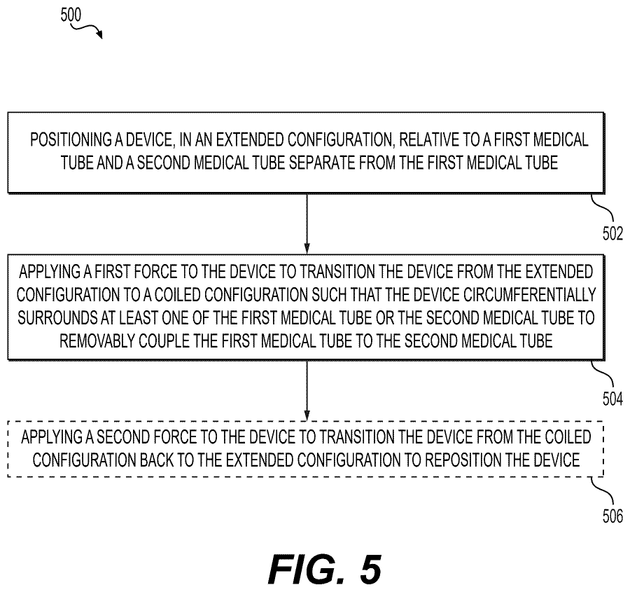

FIG. 5 depicts an example method 500 for removably coupling medical tubes. At step 502, method 500 may include positioning a device, in an extended configuration, relative to a first medical tube and a second medical tube separate from the first medical tube. The device may be one of first tube management device 116, second tube management device 200, third tube management device 300, or fourth tube management device 400. In the extended configuration, the device may be substantially linear along a longitudinal axis of the device. As one non-limiting example, first medical tube may include shaft 106 of first medical device 102, and second medical tube may include tube 114 of second medical device 112, as described in detail with reference to FIG. 1A.

When device is first tube management device 116, the device may be positioned at a desired location along a length of the first medical tube and/or the second medical tube when the first medical tube and the second medical tube are in proximity to one another (e.g., are substantially parallel to one another along the length). When device is one of second tube management device 200, third tube management device 300, or fourth tube management device 400, positioning of the device may include receiving one of the first medical tube or the second medical tube within a channel of the device (e.g., one of channel 204, channel 304, or channel 404, respectively) to couple the respective medical tube to the device. The coupled device and medical tube may then be positioned relative to the other of the first medical tube or the second medical tube.

At step 504, method 500 may include applying a first force to the device to transition the device from the extended configuration to a coiled configuration such that the device circumferentially surrounds at least one of the first medical tube or the second medical tube to removably couple the first medical tube to the second medical tube. In the coiled configuration, the device may be substantially curved to enable the device to circumferentially surround the first medical tube and/or the second medical tube. When device is first tube management device 116 or second tube management device 200, the device may circumferentially surround both the first medical tube and the second medical tube. When device is third tube management device 300 or fourth tube management device 400, the device may circumferentially surround only one of the first medical tube or the second medical tube.

An example first force applied may include contacting one end of device against first medical tube and/or second medical tube with sufficient force (e.g., by slapping against). Another example first force applied may include applying a bending action (e.g., by pushing inward) to one or both ends of the device to bend interiorly.

At optional step 506, method 500 may optionally include applying a second force to the device to transition the device from the coiled configuration back to the extended configuration to reposition. An example second force applied may include a pulling action on both ends of the device to lift the ends outward and away from the first medical tube and second medical tube until a length of the device from end to end extends along a same plane.

The method 500 described above is provided merely as an example, and may include additional, fewer, different, or differently arranged steps than depicted in FIG. 5. For example, when the device is third tube management device 300 or fourth tube management device 400, the method may alternatively include: positioning the device relative to one of the first medical tube or the second medical tube; applying the first force to the device to transition the device from the extended configuration to the coiled configuration such that the device circumferentially surrounds the one of the first medical tube or the second medical tube; and receiving the other of the first medical tube or the second medical tube via the channel of the device to secure the first and second medical tube to one another.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A device for tube management, the device comprising:
a base including a structure configured to transition the device between an extended configuration and a coiled configuration upon receiving an application of a force to the device; and
a coating applied to the base,
wherein, in the extended configuration, the device includes a channel configured to receive a tube, and
wherein, in the coiled configuration, the device is configured to circumferentially surround a medical device shaft to removably couple the tube to the medical device shaft.

2. The device of claim 1, wherein the extended configuration is a substantially linear configuration, and wherein the coiled configuration is a substantially curved configuration.

3. The device of claim 1, wherein the channel is a recess in an interior surface of the device.

4. The device of claim 3, wherein, in the coiled configuration, the device is configured to circumferentially surround the medical device shaft and the tube received within the channel.

5. The device of claim 1, wherein the channel protrudes from an exterior surface of the device.

6. The device of claim 5, wherein the channel is a partially opened channel.

7. The device of claim 6, wherein the partially opened channel includes an opening configured to receive the tube that is formed from a pair of arms extending outwardly and perpendicularly from the exterior surface of the device.

8. The device of claim 5, wherein the channel is an enclosed channel.

9. The device of claim 8, wherein the enclosed channel includes a tubular member extending outwardly and substantially perpendicularly from the exterior surface of the device, and wherein the tubular member includes a tubular member lumen configured to receive the tube.

10. The device of claim 5, wherein the channel is configured to receive the tube while the device is in the extended configuration and in the coiled configuration.

11. The device of claim 1, wherein the coating includes a first coating and a second coating different from the first coating.

12. The device of claim 11, wherein the first coating is applied to one or more surfaces of the base configured to be exposed to a body lumen when the medical device shaft to which the tube is removably coupled via the device is partially inserted into the body lumen.

13. The device of claim 11, wherein the second coating is applied to one or more surfaces of the base configured to contact at least one of the medical device shaft or the tube.

14. The device of claim 1, wherein, when the device is in the coiled configuration, the structure of the base is configured to transition the device from the coiled configuration back to the extended configuration to enable repositioning of the device relative to at least one of the medical device shaft or the tube.

15. The device of claim 1, wherein the base is comprised of metal, and wherein the coating is comprised of rubber, plastic, or a combination of rubber and plastic.

16. A medical system, comprising:
a first medical tube;
a second medical tube separate from the first medical tube; and
a tube management device configured to removably couple the first medical tube to the second medical tube, wherein the tube management device comprises:
a base including a structure configured to transition the tube management device between an extended configuration and a coiled configuration; and
a coating applied to the base,
wherein, in the coiled configuration, the tube management device is configured to circumferentially surround the first medical tube and the second medical tube to removably couple the first medical tube to the second medical tube.

17. The medical system of claim 16, wherein the tube management device further includes a channel configured to receive one of the first medical tube or the second medical tube, and wherein the channel is a recess in an interior surface of the tube management device.

18. The medical system of claim 16, wherein, when the tube management device is in the coiled configuration, the structure of the base is configured to transition the tube management device from the coiled configuration back to the extended configuration to enable repositioning of the tube management device relative to at least one of the first medical tube or the second medical tube.

19. A method for removably coupling medical tubes, the method comprising:
positioning a device relative to a first medical tube and a second medical tube separate from the first medical tube, wherein the device is in an extended configuration; and
applying a first force to the device to transition the device from the extended configuration to a coiled configuration such that the device circumferentially surrounds at least one of the first medical tube or the second medical tube to removably couple the first medical tube to the second medical tube.

20. The method of claim 19, wherein positioning the device further comprises:
receiving one of the first medical tube or the second medical tube within a channel of the device to couple the one of the first medical tube or the second medical tube to the device; and
positioning the device coupled to the one of the first medical tube or the second medical tube relative to the other of the one of the first medical tube or the second medical tube.
